(19) United States
(12) Patent Application Publication
(10) Pub. No.: US 2025/0197923 A1
(43) Pub. Date: Jun. 19, 2025

(54) DNA Polymerases and Related Methods

(71) Applicant: New England Biolabs, Inc., Ipswich, MA (US)

(72) Inventors: Heidi Crosby, Ipswich, MA (US); Burcu B. Minsky, Ipswich, MA (US); Jennifer Ong, Salem, MA (US); Gregory Patton, Peabody, MA (US); Nathan Tanner, West Newbury, MA (US); Dong Ma, Ipswich, MA (US); Sean R. Johnson, Beverly, MA (US); Andrew Gray, Beverly, MA (US)

(73) Assignee: New England Biolabs, Inc., Ipswich, MA (US)

(21) Appl. No.: 18/983,019

(22) Filed: Dec. 16, 2024

Related U.S. Application Data
(60) Provisional application No. 63/610,498, filed on Dec. 15, 2023.

(51) Int. Cl. C12Q 1/6853; C12N 9/12

(52) U.S. Cl. CPC C12Q 1/6853; C12N 9/1276; C12Y 207/07049

(57) ABSTRACT

Provided herein is an engineered DNA polymerases containing an amino acid sequence selected from: an amino acid sequence that is at least 80% identical to an amino acid sequence selected from: SEQ ID NO:4, SEQ ID NO:5, SEQ ID NO:6, and SEQ ID NO:24; and an amino acid sequence that is at least 92% identical to an amino acid sequence selected from: SEQ ID NO:2 and SEQ ID NO:3. Also provided are methods employing the described DNA polymerases.

### CROSS-REFERENCING

This application claims the benefit of U.S. provisional application Ser. No. 63/610,498, filed on Dec. 15, 2023, which application is incorporated by reference herein.

### SEQUENCE LISTING

This application is filed with a Sequence Listing in electronic form as a Sequence Listing XML, “NEB-474.xml.” created on Nov. 27, 2024, and having a size of 27,332 bytes. The contents of the Sequence Listing XML are incorporated by reference herein in their entirety.

### BACKGROUND

Sequence-specific DNA amplification is widely used in research and medical diagnostics. The most common approaches for amplifying DNA strands are polymerase chain reaction (PCR) and isothermal amplification. PCR relies upon exposure of the samples to periods of high temperature in a thermal cycling instrument to separate the strands of DNA duplexes that are formed when the polymerase copies the template strand. Strand separation is necessary to free the newly generated DNA strands so that they can be copied to achieve exponential amplification. Bst DNA polymerases are enzymes that can copy DNA or RNA strands and have a robust ability to separate the resulting duplexes and displace the upstream strand during synthesis. This strand displacing capability removes the need for high temperature strand separation and enables constant temperature (isothermal) amplification. Tests employing isothermal amplification, such as LAMP tests, are low-cost compared to PCR because they do not require a thermal cycler or other expensive equipment. Isothermal amplification techniques continue to gain popularity following their widespread adoption for COVID detection, and there is a need for continued broadening of constant temperature test capabilities. Thus, it is desirable to have engineered Bst-like DNA polymerases with enhanced functionality, such as fast reaction times, low background activity, and thermal stability.

### SUMMARY

Provided herein are engineered DNA polymerases containing an amino acid sequence selected from: an amino acid sequence that is at least 80% identical to an amino acid sequence selected from: SEQ ID NO:4, SEQ ID NO:5, SEQ ID NO:6, and SEQ ID NO:24; and an amino acid sequence that is at least 92% identical to an amino acid sequence selected from: SEQ ID NO:2 and SEQ ID NO:3. Also provided are methods employing the described DNA polymerases. The methods involve incubating a reaction mixture containing (i) a DNA polymerase; (ii) a target nucleic acid; (iii) dNTPs; and (iv) one or more primers, under conditions suitable for polynucleotide extension of the target nucleic acid to produce copied DNA product. Further provided are compositions containing such a DNA polymerase, as well as kits containing such a DNA polymerase and one or more components for carrying out a polynucleotide extension reaction.

The DNA polymerases described herein have enhanced functionality relative to commercially available strand-displacing DNA polymerases, such as fast reaction times, low background activity, and thermal stability.

### BRIEF DESCRIPTION OF THE DRAWINGS

FIG. 1 shows activities of exemplary embodiments of DNA polymerases SDpol-1, SDpol-2, SDpol-3, SDpol-4, SDpol-5 and SDpol-6 compared to commercially available enzyme in a LAMP assay, where amplified lambda DNA target is depicted by filled circles and no template control (NTC) is depicted by open circles.

FIG. 2 shows thermostability of exemplary embodiments of DNA polymerases using a LAMP assay as a read-out, where activity was maintained after heat challenges of up to 85° C.

FIG. 3 shows LAMP assay performance of exemplary embodiments of DNA polymerases including an embodiment of SDpol-1 and fusion proteins with DNA binding domains Sso7d, BD007, BD023, BD009, BD062, BD093, BD109, BD006, and BD012, where amplified lambda DNA target is depicted by filled circles and no template control is depicted by open circles.

FIG. 4 shows a comparison of exemplary embodiments of DNA polymerases BD009-SD-pol1 and Sso7d-SDpol-1 with commercially available enzymes Bst 2.0 and Bst 3.0, in a fluorescence LAMP assay, indicating time to detection of amplified human genomic DNA.

FIGS. 5A and 5B show a comparison of exemplary embodiments of DNA polymerases BD009-SD-pol1 and Sso7d-SDpol-1 with commercially available enzyme Bst 2.0, in a colorimetric LAMP assay, indicating amplification of target, where a grey scale hue below 100 arbitrary units (red sample color) indicates no amplification, and a grey scale hue above 100 arbitrary units indicates amplification (yellow sample color). FIG. 5A shows hue values prior to amplification; FIG. 5B shows hue values after 60 minutes of amplification.

### DESCRIPTION

This disclosure provides, among other things, DNA polymerases that have improvements in one or more properties. For example, the present DNA polymerases are believed to be more heat tolerant relative to commercially available Bst enzymes.

Although embodiments of the disclosure are explained in detail, it is to be understood that other embodiments are contemplated. Accordingly, it is not intended that the invention is limited in its scope to the details of construction and arrangement of components set forth in the following description or illustrated in the drawings. The invention is capable of other embodiments and of being practiced or carried out in various ways. Also, in describing the embodiments, specific terminology will be resorted to for the sake of clarity.

It must also be noted that, as used in the specification and the appended claims, the singular forms “a,” “an” and “the” include plural referents unless the context clearly dictates otherwise. For example, reference to a sheet or portion is intended also to include the manufacturing of a plurality of sheets or portions. References to a sheet containing “a” constituent is intended to include other constituents in addition to the one named.

All cited publications are incorporated by reference herein.

Also, in describing the embodiments, terminology will be resorted to for the sake of clarity. It is intended that each term contemplates its broadest meaning as understood by those skilled in the art and includes all technical equivalents which operate in a similar manner to accomplish a similar purpose.

Ranges can be expressed herein as from “about” or “approximately” one particular value and/or to “about” or “approximately” another particular value. When such a range is expressed, another embodiment includes from the one particular value and/or to the other particular value. “Comprising” or “containing” or “including” mean that at least the named compound, element, particle, or method step is present in the composition or article or method, but does not exclude the presence of other compounds, materials, particles, method steps, even if the other such compounds, material, particles, method steps have the same function as what is named.

The term “non-naturally occurring” used in reference to a polypeptide or composition described herein means that the polypeptide or composition does not exist in nature. A “non-naturally occurring” composition can differ from naturally occurring compositions in one or more of the following respects: (a) having components that are not combined in nature; (b) having components in concentrations not found in nature; (c) omitting one or components otherwise found in naturally occurring compositions; (d) having a form not found in nature, e.g., dried, freeze dried, crystalline, micellular, aqueous; and (e) having one or more additional components beyond those found in nature (e.g., buffering agents, a detergent, a dye, a solvent or a preservative). The DNA polymerases described herein are examples of non-naturally occurring polypeptides, and the compositions comprising the DNA polymerases, including those created when performing the related methods, are examples of non-naturally occurring compositions.

This disclosure relates to strand-displacing DNA polymerases designed using algorithmic strategies. As background, strand displacing polymerases, exemplified by the well-known Bst polymerase, belong to the “A” family of DNA polymerases, which in their native state contain an N-terminal 5′-3′ exonuclease domains upstream of a 3′-5′ exonuclease domain and a polymerase domain. The 5′-3′ exonuclease activity can be eliminated by mutations or truncations while maintaining the DNA polymerase activity (see, e.g., Riggs et al., Biochim Biophys Acta 1996; 1307:178-86). The crystal structure of a truncated Bst polymerase from Bacillus stearothermophilus has been solved (see, e.g., Kiefer J R et al, Structure 1997; 5:95-108) and many mutations in Bst polymerases are known (see, e.g., Oscorbin et al. Comput Struct Biotechnol J. 2023 Sep. 12; 21:4519-4535). Variants of a DNA polymerase described herein can thus be designed using sequence alignments and published structural information.

In various embodiments, provided herein are DNA polymerases comprising an amino acid sequence selected from: an amino acid sequence that is at least 80% identical to an amino acid sequence selected from: SEQ ID NO:4, SEQ ID NO:5, SEQ ID NO:6, and SEQ ID NO:24; and an amino acid sequence that is at least 92% identical to an amino acid sequence selected from: SEQ ID NO:2 and SEQ ID NO:3.

In an embodiment, the DNA polymerase includes an amino acid sequence selected from amino acid sequences at least 80% identical to an amino acid sequence selected from: SEQ ID NO:4, SEQ ID NO:5, SEQ ID NO:6, and SEQ ID NO:24. In an embodiment, the DNA polymerase can comprise an amino acid sequence at least 85% identical, at least 88% identical, at least 90% identical, at least 92% identical, at least 93% identical, at least 95% identical, at least 96% identical, at least 97% identical, at least 98% identical or at least 99% identical to any of SEQ ID NOS: 4-6. In an embodiment, the DNA polymerase comprises an amino acid sequence that is identical to SEQ ID NO: 4, SEQ ID NO:5, SEQ ID NO:6 or SEQ ID NO:24.

In an embodiment, the DNA polymerase includes an amino acid sequence selected from amino acid sequences at least 92% identical to SEQ ID NO:2, at least 93% identical, at least 95% identical, at least 96% identical, at least 97% identical, at least 98% identical or at least 99% identical to SEQ ID NO:2. In an embodiment, the DNA polymerase comprises an amino acid sequence that contains five or fewer amino acid substitutions, four or fewer amino acid substitutions, three or fewer amino acid substitutions, two or fewer amino acid substitutions, or one amino acid substitution relative to SEQ ID NO:2. In an embodiment, the DNA polymerase comprises an amino acid sequence identical to SEQ ID NO:2. The DNA polymerase can contain one or more of the following amino acids, which are present in SEQ ID NO:2 and different from the corresponding stretch of amino acids of a truncated version of the wild type Bst NCBI Reference Sequence: WP_108438052.1 (provided herein as SEQ ID NO:1): M at position 1, E at position 4, I at position 11, E at position 17, T at position 18, E at position 27, S at position 28, Q at position 41, L at position 45, F at position 47, H at position 55, I at position 58, P at position 59, T at position 60, V at position 62, S at position 66, A at position 68, R at position 70, E at position 74, V at position 82, I at position 89, H at position 95, K at position 100, I at position 102, D at position 103, S at position 116, E at position 117, S at position 118, N at position 119, T at position 132, Q at position 135, E at position 139, A at position 148, V at position 149, N at position 153, V at position 154, K at position 164, Y at position 167, K at position 170, E at position 171, T at position 172, M174I at position 174, E at position 176, K at position 178, E at position 179, Y at position 183, E at position 184, M at position 191, S at position 194, H at position 195, K at position 206, E at position 210, Q at position 213, E at position 217, E at position 225, E at position 236, T at position 237, I at position 259, I at position 283, Q at position 309, T at position 331, E at position 357, V at position 360, N at position 379, Q at position 389, D at position 407, S at position 434, G at position 438, Q at position 453, D at position 464, E at position 525, R at position 531, K at position 558, N at position 566.

In an embodiment, a DNA polymerase includes an amino acid sequences that is at least 92% identical, at least 93% identical, at least 95% identical, at least 96% identical, at least 97% identical, at least 98% identical or at least 99% identical to SEQ ID NO:3. In an embodiment, the DNA polymerase comprises an amino acid sequence identical to SEQ ID NO:3. In an embodiment, the DNA polymerase comprises an amino acid sequence that contains five or fewer amino acid substitutions, four or fewer amino acid substitutions, three or fewer amino acid substitutions, two or fewer amino acid substitutions, or one amino acid substitution relative to SEQ ID NO:3. In an embodiment, the DNA polymerase can contain one or more of the following amino acids, which are present in SEQ ID NO:3 and different from the corresponding stretch of amino acids of a wild type Bst NCBI Reference Sequence: GCD81562.1 (provided herein as SEQ ID NO:7): M at position 292, E at position 293, P at position 294, T at position 296, E at position 300, S at position 302, K at position 304, E at position 307, I at position 309, E at position 312, T at position 315, E at position 327, N at position 343, V at position 358, K at position 360, E at position 361, T at position 367, A at position 381, I at position 387, K at position 390, D at position 393, T at position 422, D at position 423, K at position 460, Q at position 465, E at position 466, K at position 468, I at position 516, K at position 519, Q at position 522, E at position 575, K at position 599K, V at position 643, E at position 647, A at position 653, A at position 675, D at position 680, E at position 696, G at position 728, E at position 739, E at position 753, E at position 754, D at position 758, A at position 814, E at position 818, Q at position 823, E at position 841, K at position 848.

With reference to an amino acid, “position” refers to the place such amino acid occupies in the primary sequence of a polypeptide numbered from its amino terminus to its carboxy terminus. A position in one primary sequence can correspond to a position in a second primary sequence, for example, where the two positions are opposite one another when the two primary sequences are aligned using an alignment algorithm (e.g., BLAST (Journal of Molecular Biology. 215 (3): 403-410) using default parameters (e.g., expect threshold 0.05, word size 3, max matches in a query range 0, matrix BLOSUM62, Gap existence 11 extension 1, and conditional compositional score matrix adjustment) or custom parameters). An amino acid position in one sequence can correspond to a position within a functionally equivalent motif or structural motif that can be identified within one or more other sequence(s) in a database by alignment of the motifs. Analogously, with reference to a nucleotide, “position” refers to the place such nucleotide occupies in the nucleotide sequence of an oligonucleotide or polynucleotide numbered from its 5′ end to its 3′ end.

In an embodiment, a DNA polymerase described herein can be a fusion protein. As used herein, the term “fusion protein” means a non-naturally occurring polypeptide containing two or more amino acid segments that are not joined in their naturally occurring states. A fusion protein can be constructed for a variety of purposes, such as for ease of purification (e.g., poly-His, chitin binding domain, maltose binding protein, glutathione S-transferase (GST), alpha mating factor or SNAP-Tag® (New England Biolabs, Ipswich, MA)); for detection (e.g., a fluorescent protein for direct detection, an enzyme for indirect detection such as horse radish peroxidase); for protein translocation within a cell, tissue or organism; for protein interaction with other targets (e.g., DNA binding domain, which can be non-specific or specific); for chemical modification (e.g., to introduce a modification site). Other kinds of functional domains can also be joined to a DNA polymerase amino acid sequence described herein. A DNA polymerase described herein can be joined with such domains at its N-terminus, C-terminus, and or the middle portion, or at more than one location. Segments of a fusion protein can optionally be separated by a linker. Polypeptide components of a fusion protein can be joined by one or more peptide bonds, disulfide linkages, and/or other covalent bonds.

Therefore, provided herein are DNA polymerases that are fusion proteins, comprising a DNA polymerase described herein joined to an exogenous amino acid sequence. In an embodiment, the exogenous amino acid sequence comprises a purification tag. Fusion proteins of the subject DNA polymerases with poly-His purification tags are described, for example, in Example 1.

In an embodiment, the exogenous amino acid sequence comprises a DNA binding protein domain. In particular embodiments, the DNA binding protein domain can be a DNA binding protein domain listed in Table 1 (see, e.g., US 2016/0160193). Example 3 describes a variety of subject DNA polymerases that are N-terminal fusions with DNA binding protein domains, including Sso7d, BD007, BD023, BD009, BD062, BD093, BD109, BD006, and BD012. In an embodiment, the exogenous amino acid sequence comprises another type of functional domain, such as those described herein above.

TABLE 1
DNA binding domains

| Name | Abbreviation | Accession |
|---|---|---|
| DNA-binding protein Tfx | BD-51 | gi\|499321160 |
| AbrB/MazE/MraZ-like | BD-52 | gi\|499321199 |
| “Winged helix” DNA-binding domain | BD-54 | gi\|499322061 |
| lambda repressor-like DNA-binding domains | BD-63 | gi\|499322443 |
| Resolvase-like | BD-67 | gi\|499322676 |
| “Winged helix” DNA-binding domain | BD-71 | gi\|499322676 |
| “Winged helix” DNA-binding domain | BD-74 | gi\|499322255 |
| “Winged helix” DNA-binding domain | BD-75 | gi\|499322388 |
| “Winged helix” DNA-binding domain | BD-81 | gi\|499322131 |
| “Winged helix” DNA-binding domain | BD-82 | gi\|499321342 |
| “Winged helix” DNA-binding domain | BD-85 | gi\|499321130 |
| “Winged helix” DNA-binding domain | BD-86 | gi\|499322705 |
| “Winged helix” DNA-binding domain | BD-88 | gi\|499320855 |
| “Winged helix” DNA-binding domain | BD-89 | gi\|499322250 |
| “Winged helix” DNA-binding domain | BD-91 | gi\|499321633 |
| “Winged helix” DNA-binding domain | BD-92 | gi\|490170077 |
| “Winged helix” DNA-binding domain | BD-94 | gi\|499320919 |
| “Winged helix” DNA-binding domain | BD-97 | gi\|499320853 |
| “Winged helix” DNA-binding domain | BD-98 | gi\|499321734 |
| “Winged helix” DNA-binding domain | BD-100 | gi\|499322439 |
| “Winged helix” DNA-binding domain | BD-102 | gi\|499322707 |
| HCP-like | BD-02 | gi\|351675391 |
| Helix-turn-helix domain, rpiR family | BD-03 | gi\|500479591 |
| Helix-turn-helix domain, rpiR family | BD-04 | gi\|15643984 |
| Bacterial regulatory proteins, lacl family | BD-08 | gi\|15643974 |
| Bacterial regulatory proteins, lacl family | BD-11 | gi\|500480095 |
| “Winged helix” DNA-binding domain | BD-14 | gi\|15644350 |
| “Winged helix” DNA-binding domain | BD-16 | gi\|24159093 |
| “Winged helix” DNA-binding domain | BD-18 | gi\|15643139 |
| “Winged helix” DNA-binding domain | BD-24 | gi\|15643159 |
| “Winged helix” DNA-binding domain | BD-30 | gi\|15643333 |
| “Winged helix” DNA-binding domain | BD-32 | gi\|15643055 |
| “Winged helix” DNA-binding domain | BD-37 | gi\|15643827 |
| “Winged helix” DNA-binding domain | BD-43 | gi\|15643699 |
| Homeodomain-like | BD-45 | gi\|15643788 |

The DNA polymerases described herein, including the fusion proteins, can have one or more activities selected from strand-displacing DNA polymerase activity, and strand-displacing DNA polymerase activity with reverse transcriptase activity. In an embodiment, the polypeptide has strand-displacing DNA polymerase activity. In an embodiment, the DNA polymerase has strand-displacing polymerase activity and reverse transcriptase activity. The DNA polymerases described herein are believed to not have substantial 5′-3′ exonuclease activity.

The DNA polymerases described herein can be thermostable relative to known Bst polymerases. As such, a DNA polymerase described herein can retain its desired activity at temperatures of over 65° C., over 70° C., over 75° C., over 80° C., and over 85° C. As non-limiting examples, such DNA polymerases can be present in a reaction mixture that is exposed to heat, e.g., when input target nucleic acid is heat denatured for binding to primers, when sample is treated to inactivate nucleases, cell lysis, and other steps facilitated by heat treatment.

The DNA polymerases described herein can be used for any purpose in which their activity is necessary or desired (generally, their DNA polymerase activity, and for some applications, their strand-displacing DNA polymerase activity, and optionally for some applications, reverse transcriptase activity). Accordingly, provided herein are methods, which involve incubating a reaction mixture containing (i) a DNA polymerase; (ii) a target nucleic acid; (iii) dNTPs; and (iv) one or more primers, under conditions suitable for polynucleotide extension of the target nucleic acid to produce copied DNA product (e.g., presence of divalent cations). In an embodiment, the conditions can be isothermal. In an embodiment, the DNA polymerase comprises an amino acid sequence selected from: an amino acid sequence that is at least 80% identical to an amino acid sequence selected from: SEQ ID NO:4, SEQ ID NO:5, SEQ ID NO:6, and SEQ ID NO:24; and an amino acid sequence that is at least 95% identical to an amino acid sequence selected from: SEQ ID NO:2 and SEQ ID NO:3.

As used herein, the term “target nucleic acid” means the substrate for a DNA polymerase described herein. As used herein, the term “nucleic acid” means a polymeric form of nucleotides of any length, such as deoxyribonucleotides or ribonucleotides, or analogs thereof. For example, a nucleic acid can be DNA, RNA or the DNA product of RNA subjected to reverse transcription (cDNA). Non-limiting examples of nucleic acids include coding or non-coding regions of a gene or gene fragment, loci (locus) defined from linkage analysis, exons, introns, messenger RNA (mRNA), transfer RNA, ribosomal RNA, ribozymes, cDNA, recombinant nucleic acids, branched nucleic acids, plasmids, vectors, isolated DNA of any sequence, isolated RNA of any sequence, nucleic acid probes, and primers. Other examples of nucleic acids include, without limitation, cDNA, aptamers, and peptide nucleic acids. A nucleic acid can contain modified nucleotides, such as methylated nucleotides and nucleotide analogs (“analogous” forms of purines and pyrimidines are well known in the art). If present, modifications to the nucleotide structure can be imparted before or after assembly of the polymer. A nucleic acid can be a single-stranded, double-stranded, partially single-stranded, or partially double-stranded DNA or RNA, depending on the application.

As used herein, the term “DNA polymerase” means an enzyme capable of catalyzing polynucleotide extension. A DNA polymerase can also have reverse transcriptase activity. As used herein, the term “polynucleotide extension” means the synthesis of DNA catalyzed by a DNA polymerase resulting in polymerization of individual nucleoside triphosphates using a primer as a point of initiation. Generally, a primer is hybridized to a target nucleic acid to form a primer-template complex. The primer-template complex is contacted with the DNA polymerase and nucleoside triphosphates (dNTPs) in a suitable environment to permit the addition of nucleotides to the 3′ end of the primer, thereby producing a copied DNA product complementary to at least a portion of the target nucleic acid. In strand-displacing polynucleotide extension the resulting duplexes can be separated by the DNA polymerase, as the upstream strand is displaced during DNA synthesis. This strand-displacing polynucleotide extension permits isothermal amplification of nucleic acid targets, as newly synthesized strands are liberated from duplexes and become available for copying by the polymerase.

Conditions suitable for polynucleotide extension, including strand-displacing polynucleotide extension, are known in the art (see, e.g., Sambrook et al., supra. See also Ausubel et al., Short Protocols in Molecular Biology (4th ed., John Wiley & Sons 1999)). In general, a reaction mixture for carrying out polynucleotide extension using a DNA polymerase includes dNTPs, a divalent cation (e.g., Mg²⁺, Mn²⁺, Co²⁺, Cd²⁺), one or more primers, and optionally can include an antibody, antibody-like molecule, an aptamer, or other entity to inhibit the DNA polymerase or another reaction component under selected conditions (such as temperature or salt concentration). Exemplary conditions for polynucleotide extension using Bst enzymes are widely published (e.g., U.S. Pat. No. 9,127,258B2, U.S. Pat. No. 9,963,687B2, U.S. Ser. No. 11/492,673B2). The design of aptamers is well known (see, e.g., Byun J. Life (Basel). 2021 Feb. 28; 11(3):193). A reaction mixture can have a pH range of about 6.5-10, such as about 7.5-9.0, and polynucleotide extension can be carried out at a temperature range of about 37-80° C., including about 50-70° C., and about 70° C. Higher reaction temperatures can have benefits for high GC content or structured DNA/RNA template, speed, and/or reduction of nonspecific amplification. The DNA polymerases described herein are inactivated at higher temperatures than some commercially available Bst polymerases (for example, see Example 2).

As used herein, the term “reverse transcriptase” means a DNA polymerase that can produce first-strand cDNA from an RNA template. Such enzymes are commonly referred to as RNA-directed DNA polymerases and have IUBMB activity EC 2.7.7.49. In some cases, a reverse transcriptase can generate a complementary DNA strand using either single-stranded RNA or DNA as a template, although as used herein the term “reverse transcriptase activity” refers to the ability of the DNA polymerase to generate a complementary DNA strand using an RNA template. Thus, in the context of reverse transcription, “template” means the substrate RNA for the reverse transcriptase to make a copied DNA product. An RNA template can be complex (e.g., total RNA, polyA+ RNA, mRNA, etc.) or not complex (e.g., an enriched RNA or an in vitro transcribed product).

As used herein, the term “primer” means an oligonucleotide that is capable, upon forming a duplex with a nucleic acid template, of acting as a point of initiation of polynucleotide extension and being extended from its 3′ end along the template so that copied DNA product is formed. The sequence of nucleotides added during the extension process is determined by the sequence of the template polynucleotide. Primers are of a length compatible with their use in synthesis of copied DNA products, and can be in the range of between 6 to 100 nucleotides in length, such as 10 to 75, 15 to 60, 15 to 40, 18 to 30, 20 to 40, 21 to 50, 22 to 45, 25 to 40, and so on, more typically in the range of between 15 to 60 nucleotides long, and any length between the stated ranges. Primers are selected for a particular application (e.g., applications such WGA and MDA can use primers as small as 6-mers; SDA can use primers in the range of 40-mers; LAMP can use primers of about 17 to 50) and are usually single-stranded and contain a 3′ hydroxyl group. A primer can be a DNA oligonucleotide, RNA oligonucleotide, or hybrid.

A primer used for polynucleotide extension using a DNA polymerase can contain a feature (e.g., chemical moiety, sequence, modified nucleotide, etc.) for the detection or immobilization of the primer so long as such feature(s) do not destroy the ability of the primer to act as a point of initiation of DNA synthesis. For example, primers can contain an additional nucleic acid sequence at the 5′ end that does not hybridize to the target nucleic acid, but that facilitates cloning or sequencing of the amplified product or introduces a site to facilitate exponential amplification (e.g., T7 RNA polymerase promoter or nicking enzyme recognition site). A primer can include conventional nucleotides, unconventional nucleotides (e.g., ribonucleotides or labeled nucleotides), nucleotide analogs, and mixtures thereof, as suitable for a particular application.

Among the uses of DNA polymerases for polynucleotide extension are isothermal DNA amplification approaches that rely on the strand displacement activity of the DNA polymerase. The term “isothermal” as used herein means a constant temperature, as opposed to cycling between temperatures. Such isothermal amplification methods include strand displacement amplification (SDA) (see, e.g., Milla et al, Biotechniques 1998; 24:392-6), linear target isothermal multimerization and amplification (LIMA) (see, e.g., Hafner et al., Biotechniques 2001; 30: 852-6), loop-mediated isothermal amplification (LAMP) (see, e.g., Notomi, et al., E63 Nucleic Acids Res 2000; 28), nicking enzyme amplification reaction (NEAR)(see, e.g., US20090081670A1); recombinase polymerase amplification (RPA) (see, e.g., Piepenburg et al., PLoS Biol. 2006; 4(7):e204); recombinase-assisted amplification (RAA)(see, e.g., Chen et al., Analyst 2020; 145:440-4); whole genome amplification, Multiple-strand Displacement Amplification (MDA) (e.g., extending DNA isolated from tissue (fresh, frozen, or preserved); see, e.g., Aviel-Ronen S, et al. BMC Genomics. 2006 Dec. 12; 7:312), and HDA (helicase dependent amplification) (see, e.g., Vincent et al., EMBO J 2004; 5(8):795-800) and library prep, including whole genome amplification. Thus, in some embodiments, a DNA polymerase described herein is used in an isothermal reaction.

In some embodiments, the isothermal reaction is a LAMP reaction. LAMP reactions use several primers (generally, from four to six primers) that bind to locations on the target nucleic acid (“LAMP primers”). Thus, a method, composition or kit described herein can involve or include one or more, two or more, three or more, four or more, five or more, six or more, or a greater number of primers, e.g., for performing a one or more polynucleotide extension reactions using a described DNA polymerase (as LAMP primers and in other reaction contexts). Guidance for selecting LAMP primers, including use of online software such as NEB LAMP primer design tool, PrimerExplorer, LAMP Designer Optigene, and Premier Biosoft, is well known in the art (see, for example, Parida et al., Rev. Med. Virol, 2008, 18:407-421 and Nagamine et al. Mol. Cell Probes 2002, 16, 223-229). Variations of LAMP reactions include reverse transcription loop-mediated isothermal amplification (RT-LAMP), multiplex loop-mediated amplification (M-LAMP). RT-LAMP reactions use reverse transcriptase activity combined with DNA polymerase activity. In an embodiment, a DNA polymerase described herein can possess reverse transcriptase activity. Thus, such DNA polymerases can also be useful in RT-LAMP reactions. DNA polymerases described herein can also be used together with other reverse transcriptase enzymes for RT-LAMP reactions to detect specific RNA sequences in a sample.

Polynucleotide extension employing a DNA polymerase can be detected and/or analyzed using various detection methods. Examples include detection of labels such as dyes and dye combinations (e.g., detecting dyes associated with copied DNA products); real-time fluorescence; gel electrophoresis; AC susceptometry (see, for example, Tian et al, Biosens. Bioelectron. 2016, 86, 420-425), and turbidimetric analysis. Many dyes can be observed by eye in colorimetric detection or visual observation of fluorescence, in addition to or instead of observation by instrumentation. A variety dyes are useful for detecting products of polynucleotide extension, e.g., molecular beacons, FRET-like dyes, metallochromic indicators, such as 4-(2-pyridylazo) resorcinol (PAR), hydroxynaphthol blue, calcein, malachite green, leuco crystal violet, DNA intercalating dyes such as SYBR Green dyes, EvaGreen, Bromo-PAPS, Goldview dye, Miami Yellow, GelRed dye, SYTO dyes, and berberine. Colorimetric dyes that are pH sensitive are useful for colorimetric LAMP reactions; examples include phenol red, cresol red, m-cresol purple, bromocresol purple, neutral red, phenolphthalein, naphtholphthalein, and thymol blue; and fluorescent dyes such as 2′,7′-Bis-(2-Carboxyethyl)-5-(and-6)-carboxyfluorescein or a carboxyl seminaphthorhodafluor (e.g. SNARF-1). Generally, dyes are selected based on factors such as signal to noise, threshold time, optical set-up. LAMP sensitivity has been improved by reducing background and enhancing signal and these improvements can be used in the methods described herein. See for example: U.S. Pat. Nos. 9,121,046, 9,546,358, 9,074,249, 9,074,243, 9,157,073, and 9,127,258 in addition to U.S. Pat. Nos. 9,580,748, 9,034,606, and 10,597,647 all incorporated in entirety by reference.

Examples 1-3 describe use of fluorescent LAMP reactions; Example 4 describes use of colorimetric LAMP reactions, and Example 5 describes use of a cas/LAMP reaction with fluorescence detection.

A DNA polymerase described herein can be used in methods that employ its reverse transcriptase activity. Therefore, in some embodiments, a DNA polymerase is used in a reverse transcription reaction. In some embodiments, the reverse transcription reaction is carried out in a reaction mixture containing an RNA template, one or more primer(s), and a DNA polymerase described herein. The reaction mixture typically contains all four standard deoxyribonucleoside triphosphates (dNTPs), a buffer, and a divalent cation, and optionally can include another reverse transcriptase.

The ability of the DNA polymerases described herein to extend DNA and RNA templates is broadly useful in a wide variety of applications. Among these uses is detecting target nucleic acids in samples for research and medical diagnostics. For instance, the DNA polymerases can be used in detecting DNA or RNA in diverse samples such as samples obtained from humans, animals, plants, environments (e.g., soils, waters, vehicles, homes, hospitals, airports), and food products, to detect targets of interest such as pathogens (e.g., viruses, bacteria, fungi, parasites) and DNA in forensic and archaeological samples. Thus, as used herein, the term “sample” means a natural or man-made substance suspected of containing a target nucleic acid, such as a biological fluid, cell, tissue, or fraction thereof, food or environmental substance that can contain or be contaminated by a target nucleic acid. A sample can be derived from a prokaryote or eukaryote and therefore can include cells from, for example, animals, plants, or fungi as well as viruses. Accordingly, a sample includes a specimen obtained from one or more individuals or can be derived from such a specimen. For example, a sample can be a tissue section obtained by biopsy, or cells that are placed in or adapted to tissue culture. Exemplary samples include biological specimens such a cheek swab, nasopharyngeal swab, throat swab, nasopharynx flush through, amniotic fluid, skin biopsy, organ biopsy, tumor biopsy, blood, urine, saliva, semen, sputum, cerebral spinal fluid, tears, mucus, and the like. A sample can be further fractionated, if desired, to a fraction containing particular cell types. For example, a blood sample can be fractionated into serum or into fractions containing particular types of blood cells. If desired, a sample can be a combination of samples from an individual such as a combination of a tissue and fluid, or a combination of samples from more than one individual (e.g., pooled samples, maternal sample containing fetal nucleic acid). Prior to analysis, a sample can be processed to preserve the integrity of nucleic acid targets. Such methods include the use of appropriate buffers and/or inhibitors, including nuclease, protease, and phosphatase inhibitors, that preserve or minimize changes in the molecules in the sample, including tissue fixatives (e.g., in the case of FFPE preserved tissues).

Also provided by the present disclosure are compositions including a DNA polymerase described herein. Such a composition can include one or more DNA polymerases and one or more substances selected for purposes such as storage stability (including a substance such as a solid support, gel, or solution), detection of presence, concentration, or activity of the polymerase, and for performing a method using the polymerase (e.g., providing the DNA polymerase with other components for isothermal amplification, referenced in some instances as a reaction mixture).

A composition can contain components for polynucleotide extension (e.g., isothermal amplification of a nucleic acid target), such as dNTPs. Compositions containing dNTPs can include one, two, three of all four of dATP, dTTP, dGTP and dCTP, and can include one or more modified dNTPs, such as forms that are resistant to, or susceptible, to a particular enzymatic or chemical conversion, or that are detectable. Examples of modified dNTPs include alpha-phosphorothioate dNTPs, dUTP, dITP, labeled dNTPs such as, e.g., fluorescein- or cyanine-dye family dNTPs. A DNA polymerase composition can include any of (including one or more of) a buffer such as an enzyme storage bugger (e.g., containing a buffering agent such as Tris, MOPS, CAPS, HEPES, Bis-Tris), an excipient, a salt (e.g., NaCl, MgSO₄, KCL, (NH₄)₂SO₄), MgCl₂, CaCl₂), a protein (e.g., albumin, an enzyme, such as a UDG, a reverse transcriptase or another polymerase), a dye (e.g., for detecting the presence, concentration or activity of the DNA polymerase), a stabilizer, a detergent (for example, ionic, non-ionic, and/or zwitterionic detergents, a poloxamer), a polynucleotide such as one or more primers and/or control polynucleotides (e.g., a plasmid, linear RNA or DNA), a cell (e.g., intact, digested, or any cell-free extract), a biological sample, an antibody or aptamer (e.g., for inhibiting or otherwise affecting the activity of a DNA polymerase), a crowding agent, a reaction mixture (generally, containing some or all components for carrying out a polynucleotide extension using the DNA polymerase), a sugar (e.g., a mono, di, tri, tetra, or higher saccharide), a starch, cellulose, a glass-forming agent (e.g., for lyophilization), a lipid, an oil, aqueous solution, a support (e.g., a matrix such as a bead, filter paper, slide) and/or (non-naturally occurring) combinations thereof. Combinations can include, for example, two or more of the listed components (e.g., a salt and a buffer) or a plurality of a single listed component (e.g., two different salts or two different sugars).

In an embodiment, the DNA polymerase is provided in a solution. The solution can contain glycerol or be glycerol-free. In another embodiment, the DNA polymerase composition includes a solid support. Optionally, the DNA polymerase can be attached non-covalently (e.g., dried or lyophilized on, or associated by hybridization or other non-covalent attachment) or covalently to the solid support. In an embodiment, the solution or solid support can also include a component such as a buffering agent; a salt; a primer; an aptamer; another component for polynucleotide extension.

Also provided by the present disclosure are kits for using a DNA polymerase herein. A kit can include one or more DNA polymerases together with one or more other components useful for carrying out a method involving polynucleotide extension, such as an isothermal amplification reaction including those described herein above.

A kit can therefore contain components for polynucleotide extension of a nucleic acid target, such as dNTPs. A kit containing dNTPs can include one, two, three of all four of dATP, dTTP, dGTP and dCTP, and can include one or more modified dNTPs, such as forms that are resistant to, or susceptible, to a particular enzymatic or chemical conversion, or that are detectable. Examples of modified dNTPs include alpha-phosphorothioate dNTPs, dUTP, dITP, labeled dNTPs such as, e.g., fluorescein- or cyanin-dye family dNTPs. Examples herein describe inclusion of dUTP in LAMP reactions to reduce carryover contamination. Incorporation of dUTP by a DNA polymerase is commonly used during amplicon generation, and excision of incorporated uracil in copied DNA product and can be catalyzed by a uracil DNA glycosidase (UDG).

A kit can include a composition such as a buffer and/or reaction mixture in any convenient form, such as in solution, concentrated form, dried form, disposed in, on, or within a solid support (e.g., a tube, plate, pellet, membrane, bead). Such a composition can contain components useful for enabling use of the DNA polymerase in a particular assay format, e.g., to promote a particular aspect of the DNA polymerase enzymatic activity, a molecular interaction, a stability profile, and other desirable properties. Accordingly, a buffer and/or reaction mixture can contain one or more salts (e.g., NaCl, MgSO₄, KCl, (NH₄)₂SO₄), detergents (ionic, non-ionic, zwitterionic), poloxamers, preservatives, inhibitors of unwanted activities, crowding agents, reducing agents (e.g., DTT, TCEP), catalysts, dyes, (e.g., dyes described herein such as DNA intercalating dyes and colorimetric dyes (e.g., Bromo-PAPS, phenol red)), and other substances.

In an embodiment, the reaction mixture is suitable for receiving and extending a target nucleic acid in the presence of the DNA polymerase and one or more primers. A reaction mixture can include components useful for carrying out a particular protocol. For example, as described in US20210285065A1, which is incorporated herein by reference, a LAMP reaction can be carried out in the presence of one or more of guanidine hydrochloride, guanidine thiocyanate, guanidine chloride, guanidine sulfate, or arginine; as such a reaction mixture can contain one or more of these components.

In an embodiment, the DNA polymerase is in a form selected from: dried form, lyophilized form, and solution form, wherein the solution is optionally glycerol-free. In some embodiments, a kit includes one or more oligonucleotides that bind to a predetermined nucleic acid template, e.g., one or more primers for isothermal amplification of a target nucleic acid. Primers, if included, can be, for example, one or more isothermal amplification primers, exonuclease-resistant primers, chemically modified primers, e.g., for fluorescence or lateral flow detection, sequencing primers, or combinations thereof. In some embodiments, a kit does not include primers or includes a limited number of primers, in instances where the kit user provides primers appropriate for their selected target nucleic acid. In some embodiments, a kit includes a control, such as a control polynucleotide (e.g., a plasmid, linear RNA or DNA, control primer (e.g., rActin control). In some embodiments, a kit includes target-specific primers (e.g., to detect a pathogen). In some embodiments, a kit includes LAMP primers. A kit can contain components for carrying out a Cas/LAMP protocol, such as a cas enzyme and a guide RNA. A kit can contain an oligonucleotide probe labeled to facilitate detection, e.g., hybridization-based fluorescence or lateral flow detection.

A kit can include an aptamer, e.g., for binding to a DNA polymerase to control the conditions under which the DNA polymerase has activity (e.g., to reduce off-target amplification) or for binding to another component in the kit (e.g., another enzyme such as a reverse transcriptase). A kit can also include instructions for practicing a desired method (e.g., extending a target nucleic acid, detecting a target nucleic acid, DNA sequencing, DNA labeling) via any communication means. For example, the instructions can be printed (e.g., on paper or plastic), and/or electronic (e.g., provided on a device such as a portable drive, or remotely accessible such as on a web application, phone application, video, or voice transmission), and/or via demonstration.

A kit can include one or more other enzymes, as suitable for a particular purpose. For example, a kit for performing RT-LAMP can optionally include a reverse transcriptase in cases where the selected DNA polymerase reverse transcriptase activity is insufficient under the selected reaction conditions.

Components of a kit can be provided in a single container or compartment (e.g., for a single step use) or multiple containers or compartments (e.g., for combining, for sequential use, for parallel use, or another desired workflow). A kit can include a sample collection container, which optionally can contain a reagent, e.g., for stabilizing the sample (e.g., a poloxamer) or preparing it for assay.

In a specific embodiment, a kit includes a DNA polymerase described herein and a component, wherein the component is optionally selected from a storage buffer; a reaction mixture; a primer, an aptamer. In DNA polymerase is in a form selected from: dried form, lyophilized form, aqueous solution form. In an embodiment, the reaction mixture can contain a buffering agent and a salt. The kit can also include dNTPs, as described in more detail above. In an embodiment, the polymerase can be provided in a separate tube from the reaction mixture. In an embodiment, a reaction mixture is suitable for receiving and extending a target nucleic acid in the presence of the DNA polymerase and one or more primers. Other formats of reaction mixtures, which require addition of certain components prior to use are also provided.

A kit can contain a DNA polymerase that is thermostable at a particular temperature, as described herein above, e.g., to enable use of reaction mixtures that will contain a DNA polymerase during a heating process (e.g., heat lysis of cells, heat denaturation of nucleic acids).

### Embodiments

Embodiment 1. A DNA polymerase comprising an amino acid sequence selected from: an amino acid sequence that is at least 80% identical to an amino acid sequence selected from: SEQ ID NO:4, SEQ ID NO:5, and SEQ ID NO:6; and an amino acid sequence that is at least 95% identical to an amino acid sequence selected from: SEQ ID NO:2 and SEQ ID NO:3.

Embodiment 2. The DNA polymerase of embodiment 1, comprising an amino acid sequence that is at least 90% identical to an amino acid sequence selected from: SEQ ID NO:4, SEQ ID NO:5, and SEQ ID NO:6.

Embodiment 3. The DNA polymerase of embodiment 1 or 2, comprising an amino acid sequence that is at least 95% identical to an amino acid sequence selected from: SEQ ID NO:4, SEQ ID NO:5, and SEQ ID NO:6.

Embodiment 4. The DNA polymerase of any of embodiments 1-3, comprising an amino acid sequence that is identical to an amino acid sequence selected from: SEQ ID NO:2, SEQ ID NO:3, SEQ ID NO:4, SEQ ID NO:5 and SEQ ID NO:6.

Embodiment 5. A DNA polymerase, wherein the DNA polymerase is a fusion protein comprising an amino acid sequence of any of embodiments 1-4 and an exogenous amino acid sequence.

Embodiment 6. The DNA polymerase of embodiment 5, wherein the exogenous amino acid sequence comprises a DNA binding domain.

Embodiment 7. The DNA polymerase of embodiment 5 or 6, wherein the exogenous amino acid sequence comprises a purification tag.

Embodiment 8. A method, comprising:
incubating a reaction mixture comprising
   (i) a DNA polymerase of any embodiment above;
   (ii) a target nucleic acid;
   (iii) dNTPs; and
   (iv) one or more primers,
   under conditions suitable for polynucleotide extension of the target nucleic acid to produce copied DNA product.

Embodiment 9. The method of embodiment 8, wherein the conditions are isothermal.

Embodiment 10. The method of embodiment 9, wherein the one or more primers comprise Loop-Mediated Isothermal Amplification (LAMP) primers.

Embodiment 11. A composition, comprising a DNA polymerase of any of embodiments 1-7.

Embodiment 12. The composition of embodiment 11, further comprising a solution, wherein the solution optionally comprises glycerol.

Embodiment 13. The composition of embodiment 11, further comprising a solid support.

Embodiment 14. The composition of any of embodiments 11-13, further comprising a component selected from a buffering agent; a salt; a primer; an aptamer.

Embodiment 15. A kit, comprising:
(i) a DNA polymerase of any of embodiments 1-7 and
(ii) a component, wherein the component is optionally selected from a storage buffer; a reaction mixture; a primer, an aptamer.

Embodiment 16. The kit of embodiment 15, wherein the DNA polymerase is in a form selected from: dried form, lyophilized form, aqueous solution form.

Embodiment 17. The kit of embodiment 15 or 16, comprising a reaction mixture, wherein the reaction mixture comprises a buffering agent and a salt.

Embodiment 18. The kit of any of embodiments 15-17, wherein the kit further comprises dNTPs.

Embodiment 19. The kit of any of embodiments 15-18, wherein the DNA polymerase is provided in a separate tube from the reaction mixture.

Embodiment 20. The kit of embodiment 19, wherein the reaction mixture is suitable for receiving and extending a target nucleic acid in the presence of the DNA polymerase and one or more primers.

Embodiment 21. The kit of any of embodiments 15-20, further comprising one or more additional enzymes, optionally selected from a reverse transcriptase and a uracil DNA glycosidase (UDG).

Embodiment 22. The kit of any of embodiments 15-21, further comprising a dye.

Embodiment 23. The kit of embodiment 22, wherein the dye is selected from a fluorescent dye and a colorimetric dye.

Embodiment 24. The kit of embodiment 22, wherein the dye is a pH sensitive dye.

Embodiment 25. The kit of any of embodiments 22-24, wherein the dye is selected from Bromo-PAPS and phenol red.

The skilled artisan will understand that the figures, described above, and examples, described below, are for illustration purposes only. Neither the figures nor the examples are intended to limit the scope of the disclosed teachings in any way.

### Examples

### Example 1. Improved Performance of Synthetic DNA Polymerases in Fluorescent LAMP Assays

This example shows activities of DNA polymerases in a fluorescence LAMP assay using lambda DNA, including lower non template amplification relative to a commercially available enzyme.

DNA polymerases SDpol-1, SDpol-2, SDopol-3, SDpol-4, SDpol-5 and SDpol-6 (SEQ ID NOS: 2, 3, 4, 5, 6, and 24, respectively) were each expressed with an N-terminal His₆ tag and purified using NEBExpress Ni Spin Columns. Their activities were compared to that of BST 2.0 (New England Biolabs, Ipswich, MA) purified in the same manner.

LAMP reactions contained 20 mM Tris-HCl (pH 8.8), 10 mM (NH₄)₂SO₄, 50 mM KCl, 8 mM MgSO₄, 0.1% Tween 20, 1.4 mM dNTPs, 0.5× LAMP fluorescent dye (NEB), and 10 ng of lambda DNA template. Each reaction contained six primers at the following concentrations: 1.6 μM FIP (CAGCCAGCCGCAGCACGTTCGCTCATAGGAGATATGGTAGAGCCGC (SEQ ID NO:17)), 1.6 μM BIP (GAGAGAATTTGTACCACCTCCCACCGGGCACATAGCAGTCCTAGGGACAGT (SEQ ID NO:18)), 0.2 μM F3 (GGCTTGGCTCTGCTAACACGTT, SEQ ID NO:19), 0.2 μM B3 (GGACGTTTGTAATGTCCGCTCC (SEQ ID NO:20)), 0.4 μM LoopF (ACCATCTATGACTGTACGCC (SEQ ID NO:21)), and 0.4 μM LoopB (CTGCATACGACGTGTCT (SEQ ID NO:22)). 25 μL LAMP reactions were set up in triplicate with and without lambda DNA template and run at 65° C. in a CFX96 Touch Real-Time PCR machine (Bio-Rad), monitoring fluorescence in the SYBR channel every 15 s.

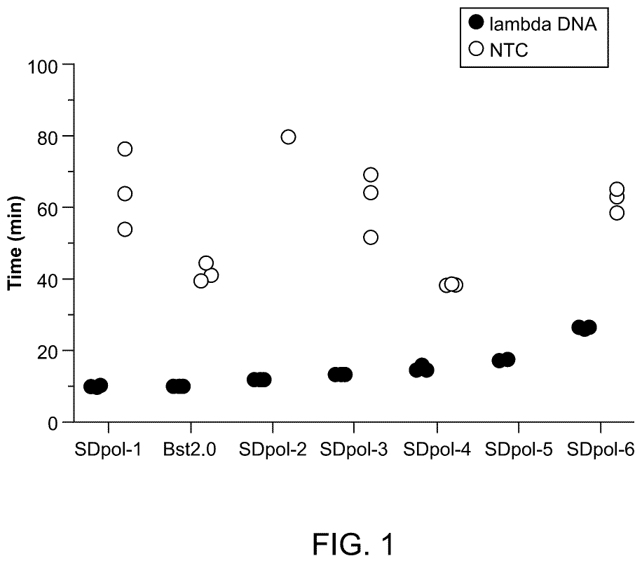

FIG. 1 shows Cq values (converted to minutes) for each enzyme, with and without lambda DNA template. SDpol-1, SDpol-2, SDpol-3, SDpol-5 and SDpol-6 amplified this target with lower non-template control amplification relative to BST 2.0, while SDpol-4 was comparable.

### Example 2. Improved Thermostability of DNA Polymerases

This example shows improved thermostability of DNA polymerases relative to a commercially available Bst enzyme in a fluorescence LAMP assay using a lambda DNA target.

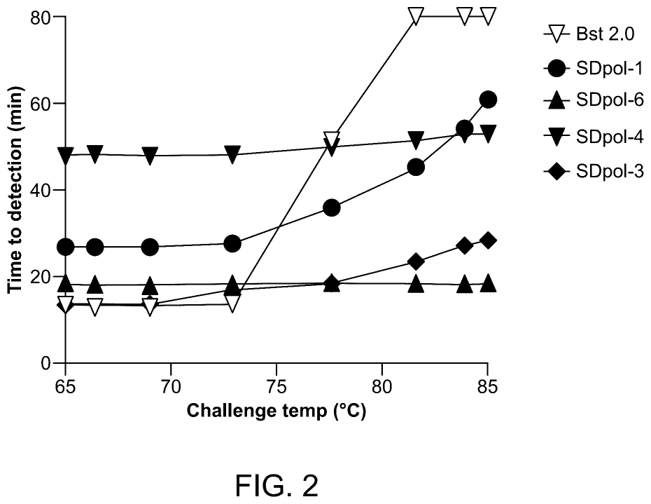

Thermostability of SDpol-1, SDpol-3, SDpol-4, SDpol-6 was demonstrated by performing one-minute heat challenges ranging from 65° C. to 85° C. before carrying out an isothermal LAMP reaction. LAMP reactions contained the same components as Example 1, except that the dNTP mix included 0.7 mM dUTP. LAMP reactions were carried out at 65° C. for 80 minutes. As shown in FIG. 2, for SDpol-1, SDpol-3, and SDpol-4, activities were unaffected by heat challenge at 65° C., 66° C., 69° C. and 73° C., and SDpol-6 activity was unaffected to 85° C.

### Example 3. DNA Polymerase Fusions with DNA Binding Domains

This example shows activities of DNA polymerase fusion proteins in a fluorescence LAMP assay using a lambda DNA target.

Fusion proteins were prepared by adding N-terminal His-tags and N-terminal DNA binding domains Sso7d, BD007, BD023, BD009, BD062, BD093, BD109, BD006, BD012, as described below, to the SDpol-1 polypeptide. Activity of these fusion proteins was compared to that of SDpol-1 alone, using the same LAMP assay as in Example 1, except the KCl concentration was increased to 100 mM.

SEQ ID NO: 8, Sso7d:
ATVKFKYKGEEKEVDISKIKKVWRVGKMISFTYDEGGGKTGRGAVSEKD
APKELLQMLEKQKK
SEQ ID NO: 9, BD007:
KRRPTINDVAKLAGVSISTVSRYLKDPSQVSEKLGERIREAIKKLGYKP
NKIAQGLRTGD
SEQ ID NO: 10, BD023:
HKKLNPKSMKRENKKMVLRYLIESGPHSRVEIARKTGLAQSAIWRIIEE
LVNEGLVEEKGTATGRRRKAVTYGPTRSFITS
SEQ ID NO: 11, BD009:
KKKYVTIRDIAEKAGVSINTVSRALNNKPDISEETRRKILKIAQELGYV
KNATASSLRSK
SEQ ID NO: 12, BD062:
NTGAQGVSEMSRMKIISVQLPQSLIHGLDALVKRGIYPNRSEAIRVAIR
ELLKKELYKEEIQEEIPEYVVK
SEQ ID NO: 13, BD093:
IINPQARLTPLELEILEIIKQKKSITITEIKEILSERRKSEYPLSLVSE
YISRLERKGYVKKIAKGRKKFVEALI
SEQ ID NO: 14, BD109:
GRKVRTQQNEILNLLNEKEKAVLRÅILEHGGEIKQEDLPELVGYSRPTI
SKVIQELENKGLIKREKSGKTFVVKIERKIKLD
SEQ ID NO: 15, BD006:
RGTVKWFDSKKGYGFITMENGEDIFVHWSAIQMDGFKTLRENETVEFEV
QKGTKGPQAVNVRPVR
SEQ ID NO: 16, BD012:
RIGEKLRKLRLSRGLTQEELAERTDLSRSFISQLESDKTSPSIDTLERI
LEALGTDLKHF

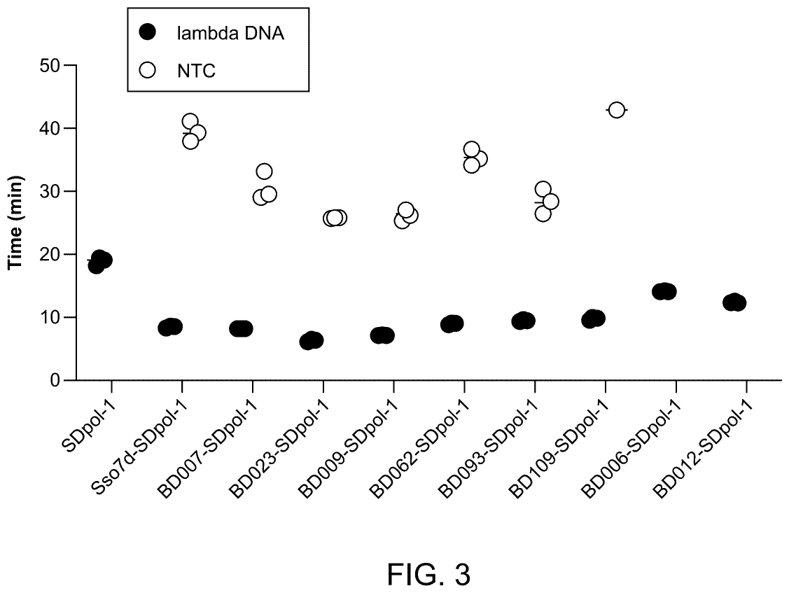

FIG. 3 shows the activities of these DNA polymerases with and without lambda DNA template. As is shown, the DNA-binding domain fusion proteins had faster performance than SDpol-1 in this LAMP assay.

### Example 4. Fast Target Amplification of DNA Polymerases in Fluorescent LAMP

This example shows activities of DNA polymerase fusion proteins in a fluorescence LAMP assay using a human genomic DNA target compared to commercially available Bst enzymes.

Two DNA polymerases (Sso7d-SDpol-1 and BD009-SDpol-1) were compared to Bst 2.0 (NEB) and Bst 3.0 (NEB) in 25 μL fluorescent LAMP reaction using a human genomic DNA target in triplicate at 10 ng, 1 ng total DNA inputs including no template control (NTC). The LAMP reaction was performed at 65° C. for 1 hour, and LAMP fluorescent dye (NEB) was spiked into each reaction to monitor the fluorescence change over time. The data was collected in a Biorad OPUS Real-Time PCR instrument monitoring fluorescence in the SYBR channel every 15 seconds, and the threshold cycle values were converted to time to detection (minutes) for reporting. Each reaction contained six primers at the following concentrations: 1.6 μM FIP 1.6 μM BIP, 0.2 μM F3, 0.2 μM B3, 0.4 μM LoopF, and 0.4 μM LoopB. The dUTP and Antarctic Thermolabile UDG (New England Biolabs) were added to the reactions to prevent carryover contamination.

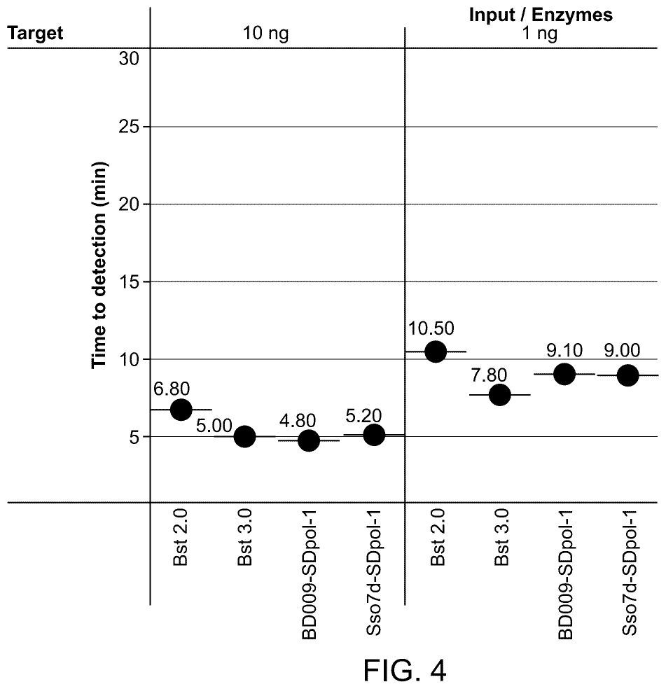

As shown in FIG. 4, Bst 3.0 was faster than Bst 2.0 to detect the DNA target, and Sso7d-SDpol-1 and BD009-SDpol-1 both showed fast amplification (lower time to detection values) similar to Bst 3.0. Sso7d-SDpol-1 and BD009-SDpol-1 showed no NTC amplification however, Bst 3.0 induced NTC amplification within 30 minutes.

### Example 5. Fast Target Amplification Using DNA Polymerases in Colorimetric LAMP

This example shows an activity comparison between Bst 2.0 and two embodiments of DNA polymerases (Sso7d-SDpol-1 and BD009-SDpol-1) in a colorimetric LAMP assay.

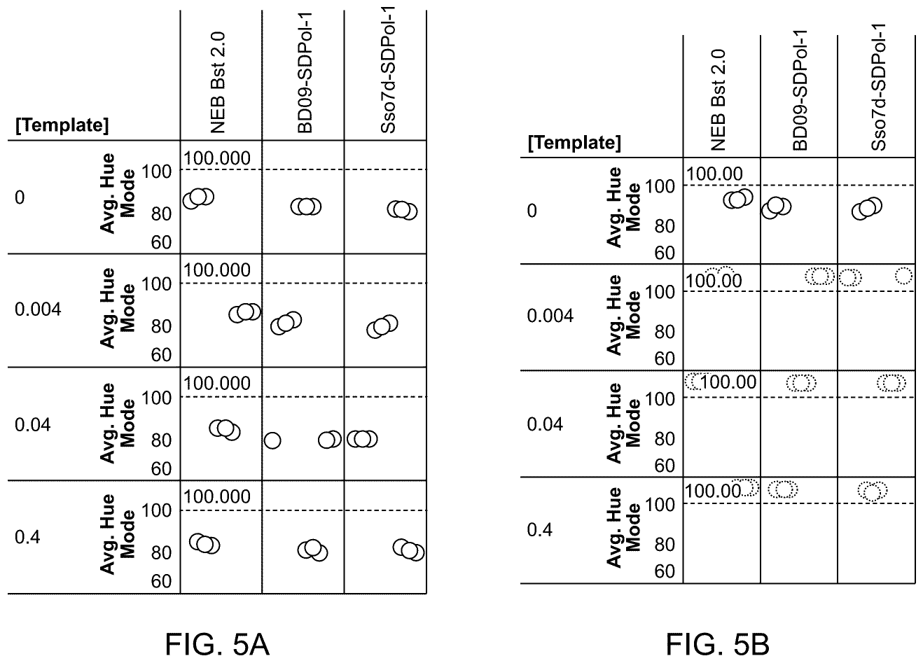

Sso7d-SDpol-1 and BD009-SDpol-1 were compared to Bst 2.0 in 25 μL LAMP reaction using a human genomic DNA target in triplicate at 10 ng, 1 ng, 0.1 ng total DNA inputs including no template control (NTC). The samples were incubated at 65° C. for 1 hour, and 5-Bromo-PAPS dye and MnCl₂ were spiked into each reaction to monitor the color change over time for visual detection. Bromo-PAPs-MnCl₂ complex produces red color in the absence of amplification. Following amplification, red color change to yellow due to precipitation of MnCl₂ by inorganic pyrophosphate byproducts. The plates were scanned before and after the reaction and, each well color was quantified by taking the mean hue of the subset of pixels associated with each well. Before and after amplification hue values (grey scale) are presented in FIGS. 5A and 5B, respectively. In the figure, hue values larger than 100 represent amplified samples (color change to yellow), and smaller than 100 represents samples with no amplification (red color). Each reaction contained six primers at the following concentrations: 1.6 μM FIP 1.6 μM BIP, 0.2 μM F3, 0.2 μM B3, 0.4 μM LoopF, and 0.4 μM LoopB. The dUTP and AT UDG were added to the reactions to prevent carryover contamination.

As shown in FIGS. 5A and 5B, Sso7d-SDpol-1 and BD009-SDpol-1 showed fast amplification, and even at low template input (0.1 ng) the sample color change to yellow as reflected in the above-100 data points in FIG. 5B. The DNA polymerases showed no NTC amplification after 1 hour.

### Example 6. Improved LAMP Detection by Cas Enzyme Collateral Activity

This example shows high performance of a DNA polymerase in a LAMP/Cas assay.

This assay is described in Joung J, et al. N Engl J Med. 2020 Oct. 8; 383(15):1492-1494. doi: 10.1056/NEJMc2026172. RT-LAMP reactions were incubated at 45-65° C. Primers targeting SARS-CoV-2 Gene N and conditions for the Bst 2.0 control reaction were taken from Joung et al, N Engl J Med 2020. Reactions were set up in 25 μL volume with 0-10000 copies of SARS-CoV-2 RNA (Control 16, Twist Biosciences) in buffer containing 20 mM Tris pH 8.8, 50 mM KCl, 10 mM (NH₄)₂SO₄, 7 mM (BD009-SDPol-1) or 8 mM (Bst 2.0) MgSO₄, 1.4 mM each dNTP, 0.1% v/v Tween-20. For real-time monitoring of amplification, reactions contained 1 μM SYTO™-82 (ThermoFisher) measured via the HEX channel of a Bio-Rad CFX96 instrument. To independently monitor amplification with a sequence-specific mechanism, reactions also contained 0.5 μM Alicyclobacillus acidiphilus Cas12b (AapCas12b), 0.5 μM guide RNA (from Joung et al; 5′-GUCUAGAGGACAGAAUUUUUCAACGGGUGUGCCAAUGGCCACUUUCCAGGUGGCAAAGCCCGUUG AGCUUCUCAAAUCUGAGAAGUGGCACCGAAGAACGCUGAAGCGCUG (SEQ ID NO:23)), and 0.2 μM reporter DNA (5′-FAM-T₁₀₋BHQ1) measured via the FAM channel. Simultaneous measurement in both channels allows for determination of amplification specificity as the SYTO-82 signal will arise from spurious nonspecific amplification while the FAM Cas12b signal is only present when the targeted sequence is amplified, confirming the correct identity of the amplified DNA. In reactions with Bst 2.0, RNA was confidently detected to 1000 copies but required >30 minutes of incubation. Reactions with BD009-SDpol-1 were significantly faster to detection down to 10 copies. Analysis of the Cas12b FAM signal allowed for specificity determination, and BD009-SDpol-1 was confirmed to be producing specific amplification product with 100 copies RNA input. LAMP time was calculated by an average of 2 replicates using the HEX threshold and Cas RFU from the FAM signal subtracting baseline signal from endpoint fluorescence. Results are shown in Table 2.

TABLE 2
LAMP/Cas activity

| | Bst 2.0 | | BD009-SDpol-1 | |
|---|---|---|---|---|
| | LAMP Time | | LAMP Time | |
| Copies | (min) | Cas RFU | (min) | Cas RFU |
| 10000 | 30.0 | 2200 | 8.7 | 5228 |
| 1000 | 38.3 | 950 | 11.7 | 5429 |
| 100 | n/a | 0 | 14.4 | 5230 |
| 10 | n/a | 0 | 18.6 | 2386 |
| NTC | n/a | 0 | 21.2 | 0 |

What is claimed is:

1. A DNA polymerase comprising an amino acid sequence selected from:
an amino acid sequence that is at least 80% identical to an amino acid sequence selected from: SEQ ID NO:4, SEQ ID NO:5, SEQ ID NO:6, and SEQ ID NO:24; and
an amino acid sequence that is at least 92% identical to an amino acid sequence selected from: SEQ ID NO:2 and SEQ ID NO:3.

2. The DNA polymerase of claim 1, comprising an amino acid sequence that is at least 90% identical to an amino acid sequence selected from: SEQ ID NO:4, SEQ ID NO:5, SEQ ID NO:6, and SEQ ID NO:24.

3. The DNA polymerase of claim 1, comprising an amino acid sequence that is at least 95% identical to an amino acid sequence selected from: SEQ ID NO: 2, SEQ ID NO:3, SEQ ID NO:4, SEQ ID NO:5, SEQ ID NO:6, and SEQ ID NO:24.

4. The DNA polymerase of claim 1, comprising an amino acid sequence that is identical to an amino acid sequence selected from: SEQ ID NO:2, SEQ ID NO:3, SEQ ID NO:4, SEQ ID NO:5 SEQ ID NO:6, and SEQ ID NO:24.

5. A DNA polymerase, wherein the DNA polymerase is a fusion protein comprising an amino acid sequence of claim 1 and an exogenous amino acid sequence.

6. The DNA polymerase of claim 5, wherein the exogenous amino acid sequence comprises a DNA binding domain.

7. The DNA polymerase of claim 5, wherein the exogenous amino acid sequence comprises a purification tag.

8. A method, comprising:
incubating a reaction mixture comprising:
(i) a DNA polymerase of claim 1;
(ii) a target nucleic acid;
(iii) dNTPs; and
(iv) one or more primers,
under conditions suitable for polynucleotide extension of the target nucleic acid to produce a copy of the target nucleic acid.

9. The method of claim 8, wherein the conditions are isothermal.

10. The method of claim 9, wherein the one or more primers comprise Loop-Mediated Isothermal Amplification (LAMP) primers.

11. A composition comprising: (i) an aqueous solution of or (ii) a freeze-dried/lyophilized form of the DNA polymerase of claim 1.

12. The composition of claim 11, wherein the DNA polymerase is in a solution that comprises glycerol.

13. The composition of claim 11, further comprising a solid support.

14. The composition of claim 11, further comprising one or more components selected from a buffering agent, a salt, a primer, and an aptamer.

15. A kit comprising:
a DNA polymerase of claim 1; and
a reaction buffer.

16. The kit of claim 15, further comprising dNTPs.

17. The kit of claim 15, wherein the DNA polymerase is in a form selected from: dried form, lyophilized form, solution form.

18. The kit of claim 15, wherein the reaction buffer comprises a buffering agent and a salt.

19. The kit of claim 15, wherein the DNA polymerase is provided in a separate tube from reaction buffer.

20. The kit of claim 15, wherein the reaction mixture is suitable for receiving and extending a target nucleic acid in the presence of the DNA polymerase and one or more primers.

21. The kit of claim 15, further comprising one or more additional enzymes, optionally selected from a reverse transcriptase and a uracil DNA glycosidase (UDG).


SEQUENCE LISTING (24 sequences)

SEQ ID NO: 1 - protein, 588 aa, Geobacillus thermodenitrificans
AAEGEKPLEEMEFAIVDVITEEMLADKAALVVEVMEENYHDAPIVGIALVNEHGRFFMRPETALADSQFLAWLADETKKKSMFDAKRAVVALKWKGIELRGVAFDLLLAAYLLNPAQDAGDIAAVAKMKQYEAVRSDEAVYGKGAKRSLPDEQTLAEHLVRKAAAIWALEQPFMDDLRNNEQDQLLTKLEQPLAAILAEMEFTGVNVDTKRLEQMGSELAEQLRAIEQRIYELAGQEFNINSPKQLGVILFEKLQLPVLKKTKTGYSTSADVLEKLAPHHEIVENILHYRQLGKLQSTYIEGLLKVVRPDTGKVHTMFNQALTQTGRLSSAEPNLQNIPIRLEEGRKIRQAFVPSEPDWLIFAADYSQIELRVLAHIADDDNLIEAFQRDLDIHTKTAMDIFHVSEEEVTANMRRQAKAVNFGIVYGISDYGLAQNLNITRKEAAEFIERYFASFPGVKQYMENIVQEAKQKGYVTTLLHRRRYLPDITSRNFNVRSFAERTAMNTPIQGSAADIIKKAMIDLAARLKEEQLQARLLLQVHDELILEAPKEEIERLCELVPEVMEQAVTLRVPLKVDYHYGPTWYDAK

SEQ ID NO: 2 - protein, 587 aa, synthetic construct
AEEEKPLEEIEFAIVETITEEMLADESALVVEVMEENYHQAPILGFALVNEHGHFFIPTEVALASSAFRAWLEDETKKKSVFDAKRAIVALKWHGIELKGIDFDLLLAAYLLNPSESNGDIAAVAKMKQYTAVQSDEEVYGKGAKRAVPDENVLAEHLVRKAKAIYALKETFIDELKENEQYELLTKLEMPLSHILAEMEFTGVKVDTERLQQMGEELAEQLREIEQRIYELAGETFNINSPKQLGVILFEKLQLPVIKKTKTGYSTSADVLEKLAPHHEIIENILHYRQLGKLQSTYIEGLLKVVRQDTGKVHTMFNQALTQTGRLSSTEPNLQNIPIRLEEGRKIRQAFVPSEEDWVIFAADYSQIELRVLAHIANDDNLIEAFQQDLDIHTKTAMDIFHVSEDEVTANMRRQAKAVNFGIVYGISDYGLSQNLGITRKEAAEFIERYFQSFPGVKQYMEDIVQEAKQKGYVTTLLHRRRYLPDITSRNFNVRSFAERTAMNTPIQGSAADIIKKAMIDLAERLKEERLQARLLLQVHDELILEAPKEEIERLCKLVPEVMENAVTLRVPLKVDYHYGPTWYDAK

SEQ ID NO: 3 - protein, 586 aa, synthetic construct
EPETALPEISYKIVEEITEEILTDEAALVVEVLEENYHKAPILGFAIANENGNFFIRTDTALSSPVFKEWLEDETKKKSVFDGKRAIVALKWQGIHLKGIDFDLLIASYLLNPSQSTEDVASIAKTKQYTDVQSDEAVYGKGAKQKVPDEQVLAEHLVRKAAAIRALKQGFIQELKENEQYSLFTDLELPLSTILAEMEFTGVKVDVERLKEMGEELAEQLKEIEQKIYQLAGQEFNINSPKQLGVILFEKLQLPVLKKTKTGYSTSAEVLEKLAPQHEIVEEILHYRQLGKLQSTYIEGLLKVVHKDTNKVHTIFNQALTQTGRLSSTEPNLQNIPIRLEEGRKIRQAFVPSEEDWVIFAADYSQIELRVLAHIANDENLIAAFRHDLDIHTKTAMDIFHVSEEEVTPNMRRQAKAVNFGIVYGISDYGLSQNLGITRKEAAEFIERYFEIFPGVKQYMEEIVQDAKQKGYVTTLLHRRRYLPDITSRNFNLRSFAERTAMNTPIQGSAADIIKKAMIDLAKRLEKENMQARMLLQVHDELILEAPKEEIERLQKIVPEVMENAVQLRVPLKVDYHFGPTWYDAK

SEQ ID NO: 4 - protein, 585 aa, synthetic construct
AEEIDFEIVTEVNQDEWEDMLTSPMALYVEMLDENYHQADILGFALANENGCYYIPTETALQSEAFREWLADPSQKKWVYDAKRTQVALKWHGIELKGITFDVLLASYLLNPSESNHDLSDIAKRYGNTSIPSDEAVYGKGAKRKVPEEDVLAEHLARKANAIYRLHPQLEEELEENELSSLFYDLELPLSLVLAEMEFTGVKVDRERLKEMGEELQERLDELTKEIYELAGTEFNINSPKQLGEILFDKLGLPVIKKTKTGYSTSADVLEKLAPQHEIVPKILHYRQLGKLQSTYIEGLLKVIHPETGKIHTRFNQTLTATGRLSSTDPNLQNIPIRLEEGRKIRQAFVPSEPDWVIFAADYSQIELRVLAHISQDENLIEAFRQDMDIHTKTAMDVFHVSEDEVTSLMRRQAKAVNFGIVYGISDYGLSQNLNITRKEAKEFIERYFESYPGVKRYMDDIVQQAKEDGYVTTLLHRRRYLPDINSRNFNLRSFAERTAMNTPIQGSAADIIKKAMVQMDERLKEENLKARMLLQVHDELIFEVPEDELETLKKLVPEVMENAIELNVPLKVDVSYGPTWYDAK

SEQ ID NO: 5 - protein, 605 aa, synthetic construct
EAELSEKDYRVVLSEEELEELVEELRKAGRFAIATATTSQDPMRAELVGISLCCEPHQAYYIPVGHRYLGAPEQLPLDQVLERLKPILEDPKIKKIGQNIKYDWIVLKRHGIELRGIVFDTMIASYLLNPSRRSHNLDDIALEYLGHKMISYKEVTGKGKKQVSFDQVPIEKATEYACEDADITLRLYHVLRPKLEEEGLQELFHEVEMPLIPVLAKMEMTGIRIDTELLRELSKEFEKRLQQLEEKIYELAGEEFNINSPQQLGEILFEKLKLPVAKKTKTGYSTDVEVLTELAQQHELPALILEYRSLSKLKSTYVDALPKLVHPETGRIHTSYNQTVTATGRLSSSDPNLQNIPIRTEEGRRIRQAFIPEPGWLLLSADYSQIELRILAHYSGDPALIEAFRRGEDIHTRTAAEIFGVPPEMVTPEMRRQAKTINFGIIYGMSAFGLSKELGISRKTAQEYIDRYFERYQGVKRYIEETIEQARKNGYVTTLLGRRRYLPDINSKNRNVRQFAERTAINTPIQGTAADLIKLAMIRIHRRLKKEGLKSRMLLQVHDELVFEVPEDELEKVKQLVREEMEGVMELKVPLKVDIGWGKNWAEAH

SEQ ID NO: 6 - protein, 610 aa, synthetic construct
AHSVADKNIDNTPHTYHLVDTPEKRAQLIQQLAQQKAFCFATATTSIDPNQAELVGMSFSIKPHEAWYVPVPADREEAQKIVEAFRPVFENEAIEKIGQNIKYDAIVLKWYGVEVKGPYFDTMIAHYLLEPELRHNMDYLAETYLKYKPVSIESLIGKKGKNQLSMRDVPLEKVKEYAAEDADITLQLKEYLEPKLEEEGLQKLYDDIEAPLIKVLVDMEYEGVRIDPDFLKEYSKELEKQIAELEQEIYEQAGVRFNIASPKQVGEVLFDRLKIPYRWKKTKTGQYSTDEDKLTELAAEYPIVADILDHRGLTKLKSTYVDALPKMVNPRTGRIHSSFNQALAATGRLSSNNPNLQNIPIRTPEGAKVREAFIPRDDDHILLSADYSQIELRLIAEISGDEAMLEAFQSGQDIHRATAARVFGVPFDEVTSEQRYRAKTVNFSIIYGAGATNLSRQLGIKRSEAKELIDQYFKQYKGLKKYMDNTVEQARKHGYVTTLMGRRRYLRDINSRNSLERSNAERMAINTPIQGTAADMIKIAMINIHQALREKKFQSKMILQVHDELVFDVHKDELEAVKPIIEEKMKNAIPNLKVPILVEMGVGKNWLEAH

SEQ ID NO: 7 - protein, 878 aa, Parageobacillus thermoglucosidasius
MLKRKLVLIDGNSVAYRAFFALPLLHNDKGIHTNAVYGFTMMLMKILEEEKPTHMLVAFDAGKTTFRHKTFEEYKGGRQKTPPELSEQFPLLRELLNAYQIRFYELENYEADDIIGTLCAKAENEGFEVKVISGDRDLTQLVSDHVTVDITKKGITDVESYTPETVREKYGLAPQQIIDLKGLMGDKSDNIPGVPGIGEKTALKLLKEFGSIENILDSIEQISGSKLKENLEKHRDLALMSKQLATILRDAPIDLSFEDIEYRGYDADKVVALFKELSFNSLLGKLAPQEEEKTEVALPKIRYEIVDEVTEAILSDEAALVVEVLESNYHKAPILGFAIANEHGNFFIRTDTALSSPLFTTWLEDEAKKKSVFDGKRAIVSLKWQGVHLRGIQFDLLIASYLLNPSQSTEDVASIAKTKQYAGVQSDEAVYGKGAKQKVPDEQVLAEHLVRKAAAIRALEQGFIHDLQENEQYSLFTDLELPLSTILAEMEFTGVKVDVERLKEMGEELAEQLKEVEQEIYRLAGQEFNINSPKQLGVILFEKLQLPVLKKTKTGYSTSAEVLEKLAPQHEIVEKILHYRQLGKLQSTYIEGLLKVVHRDTNKVHTIFNQALTQTGRLSSTEPNLQNIPIRLEEGRKIRQAFIPSEPDWVIFSADYSQIELRVLAHIANDENLIDAFRHGLDIHTKTAMDIFHVSKEEVTPNMRRQAKAVNFGIVYGISDYGLSQNLNITRKEAAEFIKRYFEIFPGVKQYMKDIVQEAKQKGYVTTLLHRRRYLPDITSRNFNLRSFAERTAMNTPIQGSAADIIKKAMIDLSKRLQKENMKARMLLQVHDELILEAPKDEIERLQQIVPEVMENAVQLRVPLKVDYHFGPTWYDAK

SEQ ID NO: 8 - protein, 63 aa, Saccharolobus solfataricus
ATVKFKYKGEEKEVDISKIKKVWRVGKMISFTYDEGGGKTGRGAVSEKDAPKELLQMLEKQKK

SEQ ID NO: 9 - protein, 60 aa, Thermotoga maritima
KRRPTINDVAKLAGVSISTVSRYLKDPSQVSEKLGERIREAIKKLGYKPNKIAQGLRTGD

SEQ ID NO: 10 - protein, 81 aa, Thermotoga maritima
HKKLNPKSMKRENKKMVLRYLIESGPHSRVEIARKTGLAQSAIWRIIEELVNEGLVEEKGTATGRRRKAVTYGPTRSFITS

SEQ ID NO: 11 - protein, 60 aa, Thermotoga maritima
KKKYVTIRDIAEKAGVSINTVSRALNNKPDISEETRRKILKIAQELGYVKNATASSLRSK

SEQ ID NO: 12 - protein, 71 aa, Pyrococcus furiosus
NTGAQGVSEMSRMKIISVQLPQSLIHGLDALVKRGIYPNRSEAIRVAIRELLKKELYKEEIQEEIPEYVVK

SEQ ID NO: 13 - protein, 75 aa, Pyrococcus furiosus
IINPQARLTPLELEILEIIKQKKSITITEIKEILSERRKSEYPLSLVSEYISRLERKGYVKKIAKGRKKFVEALI

SEQ ID NO: 14 - protein, 82 aa, Pyrococcus furiosus
GRKVRTQQNEILNLLNEKEKAVLRAILEHGGEIKQEDLPELVGYSRPTISKVIQELENKGLIKREKSGKTFVVKIERKIKLD

SEQ ID NO: 15 - protein, 65 aa, Thermotoga maritima
RGTVKWFDSKKGYGFITMENGEDIFVHWSAIQMDGFKTLRENETVEFEVQKGTKGPQAVNVRPVR

SEQ ID NO: 16 - protein, 60 aa, Thermotoga maritima
RIGEKLRKLRLSRGLTQEELAERTDLSRSFISQLESDKTSPSIDTLERILEALGTDLKHF

SEQ ID NO: 17 - DNA, 46 bp, synthetic construct
  source 1..46: mol_type = other DNA
cagccagccgcagcacgttcgctcataggagatatggtagagccgc

SEQ ID NO: 18 - DNA, 51 bp, synthetic construct
  source 1..51: mol_type = other DNA
gagagaatttgtaccacctcccaccgggcacatagcagtcctagggacagt

SEQ ID NO: 19 - DNA, 22 bp, synthetic construct
  source 1..22: mol_type = other DNA
ggcttggctctgctaacacgtt

SEQ ID NO: 20 - DNA, 22 bp, synthetic construct
  source 1..22: mol_type = other DNA
ggacgtttgtaatgtccgctcc

SEQ ID NO: 21 - protein, 20 aa, synthetic construct
ACCATCTATGACTGTACGCC

SEQ ID NO: 22 - DNA, 17 bp, synthetic construct
  source 1..17: mol_type = other DNA
ctgcatacgacgtgtct

SEQ ID NO: 23 - RNA, 111 nt, Alicyclobacillus acidiphilus
  source 1..111: mol_type = other RNA
gtctagaggacagaatttttcaacgggtgtgccaatggccactttccaggtggcaaagcccgttgagcttctcaaatctgagaagtggcaccgaagaacgctgaagcgctg

SEQ ID NO: 24 - protein, 550 aa, synthetic construct
EHEACYIPVGHTGPGAEAQLDLEKVLEALRPLLEDESPSKVGQNIKYEWVVFRRHGVELRGIGFDTMVASYLLNPGSRSHKLERIAAEHLGETMISYKDVTGKGKKQVSFAEVPVEKAAEYACEDAEVTFRLVPILRRKLEEEGLEELYDSLELPLIEVLARMELRGVRVDADKLKELSKEFDKRLQQQEERIYELAGEEFNIQSPKQLGEILFDKLKLPVVKKTKTGPSTDVSVLEELAAQHPIAEHILEYRSLAKLKNTYVDALPKLVHPETGRIHTSYNQTVTATGRLSSSDPNLQNIPVRTEEGRRIREAFIPEPGYVLLSADYSQIELRILAHYSGDENLIEAFRAGEDIHRRTAAEIFGVAPHEVTPEMRRQAKTINFGIIYGMGPFGLARQLGISQTSAKAAIDRYFERYQGVKRFIEETIEEARKRGYVQTLLGRRRYIPELQSRNRTVRQQGERLAVNTTIQGTAADLIKKAMIEIDRRLRKEGLRSQMILQVHDELVFEVPEDELERVKELVRERMEGVMPLRVPLKVDVGWGKNWAEAH